(19) United States
(12) Patent Application Publication
(10) Pub. No.: US 2005/0149186 A1
(43) Pub. Date: Jul. 7, 2005

(54) Cosmetic and reconstructive prosthesis containing a biologically compatible rupture indicator

(75) Inventors: Thomas Roballey, Huntington, CT (US); Nathan Feldman, Deerfield Beach, FL (US); Barry H. Schwibner, Boca Raton, FL (US)

Correspondence Address: St. Onge Steward Johnston & Reens LLC, 986 Bedford Street, Stamford, CT, 06905-5619, US

(73) Assignee: RFS HEALTH SCIENCES, Inc.

(21) Appl. No.: 10/918,110

(22) Filed: Aug. 13, 2004

Related U.S. Application Data
(63) Continuation-in-part of application No. 10/773,604, filed on Feb. 5, 2004.
(60) Provisional application No. 60/445,227, filed on Feb. 6, 2003.
(60) Provisional application No. 60/511,707, filed on Oct. 17, 2003.

(51) Int. Cl. A61F 2/12; A61F 2/04

(52) U.S. Cl. 623/8; 623/23.67; 128/898

(57) ABSTRACT

A prosthesis containing a rupture indicator is disclosed, which includes an indicator lumen enclosed by an indicator lumen envelope made of at least one layer of elastomer containing therein a biologically compatible chemical indicator and a carrier medium; and at least one implant lumen enclosed by an implant lumen envelope made of at least one layer of elastomer, disposed within the indicator lumen. The implant lumen contains therein an implant filling material. Also disclosed is a single lumen prosthesis which includes an envelope made of elastomer containing therein an implant filling material and a biocompatible chemical indicator in a carrier medium. Further disclosed is a method of detecting rupture of a prosthesis, which includes surgically implanting the prosthesis containing a biologically compatible chemical indicator in a desired location of a patient's body and monitoring a change of a body excretion or secretion for indication of prosthesis rupture.

### CROSS REFERENCE TO RELATED APPLICATIONS

This application is a continuation-in-part of patent application Ser. No. 10/773,604, filed on Feb. 5, 2004, which claims the benefit under 35 USC 119 (e) of the provisional patent application Ser. No. 60/445,227, filed on Feb. 6, 2003. This application also claims the benefit under 35 USC 119 (e) of the provisional patent application Ser. No. 60/511,707, filed on Oct. 17, 2003. All prior applications are herein incorporated by reference in their entirety.

### FIELD OF THE INVENTION

The present invention relates in general to the field of prosthesis for cosmetic and reconstructive surgery, and more particularly to a prosthesis, such as breast prosthesis, containing a biologically compatible chemical indicator for indicating rupture of the prosthesis.

### BACKGROUND OF THE INVENTION

Almost any part of the body can be filled to create balance and harmony. Often by adding to an area, it can affect the whole face or body. Today implants are widely used in cosmetic and reconstructive corrections. One of the commonly used substances as the filling material is silicone gel. It has been used for various facial implants, such as brow, nose, cheek, chin and lips, and various body implants, such as pectoral and breast, triceps and biceps, genitals, buttocks and calf. Among all types of cosmetic and reconstructive implants, the breast implant has the largest number of implementation, hence is addressed with specific emphasis hereinafter.

Over the last four decades, surgical breast augmentation in the United States has been primarily done by placement of breast implants. Implants are surgically placed either in front of the pectoralis major muscle—called subglandular or pre-pectoral implants—or they are placed behind the pectoralis major muscle—called submuscular, retroglandular, retropectoral, or subpectrol implants.

The type of the material in the implants and the variations in the shape and supporting shells in the implant also categorize implants. A silicone gel-filled implant is composed of silicone gel contained within a silicone polymer membrane or envelope. A saline implant refers to an implant composed of saline within a silicone polymer membrane. A double-lumen implant refers to an implant having two shells, typically an inner shell filled with silicone gel surrounded by an outer shell filled with saline. A reverse double-lumen implant refers to an inner shell of saline surrounded by silicone. Other variations have been implanted with three or more shells.

Before 1992, the majority of breast augmentation implants in the United States contained silicone gel. This was due to general acceptance by the medical community at the time, surgeons' preference, and the reported better texture and “feel” of a silicone gel-filled implant versus a saline-filled implant by the patients. It has been estimated that over one million women in the United States alone have received silicone gel-filled breast implants.

In the 1980s, independent authors questioned a possible association between silicone gel-filled implants and the subsequent development of connective-tissue diseases. Fueled by media hype and class action lawsuits, the Food and Drug Administration (FDA) was asked to analyze the data and make a decision. In 1992, the FDA announced that breast implants containing silicone gel would only be available in the United States under clinically controlled trials. It has since been primarily restricted in the United States to women undergoing post-mastectomy reconstruction and those requiring secondary surgery after breast augmentation. Saline-filled breast implants have replaced silicone gel-filled implants as the common breast prosthesis in the past decade. However, in comparison to silicone gel-filled implants, saline-filled breast implants are inferior in terms of mimicking elasticity, feel, and movement of the natural breast tissue.

Since 1992, there are many studies investigating the safety concerns of the silicone gel-filled implants. In 1999, after reviewing dozens of studies, the Institute of Medicine (IOM) concluded in its landmark 1999 report that silicone gel-filled implants do not cause the autoimmune disorders such as lupus or arthritis. The main safety concern according to the report is the implants' tendency to rupture. The silicone can bleed or leak out of its shell, causing infections, and/or local tissue reactions. The IOM 1999 report became the turning point for the breast implant industry and the plastic surgery profession, opening the door for the return of silicone gel-filled breast implants for cosmetic use.

Silicone gel-filled implant rupture is often locally symptomatic, and continues to be a genuine clinical concern for patients and physicians. In the United States, an estimated one to two million patients, or approximately 1% of the adult female population, have breast implants. The risk of implant rupture increases with the age of the implant. One recent study revealed that the median lifespan of a silicone gel-filled breast implant is 16.4 years. In that study, 79.1% of implants were intact at 10 years; the percentage decreased to 48.7% at 15 years.

Another study revealed that at least 77% of 344 women from Birmingham, Ala. who were not referred for examination had at least one implant that “ruptured” or had an “indeterminate” finding upon MRI. The reported median implant age at rupture was 10.8 years, and submuscular implants were more likely than subglandular implants to rupture.

In essentially all patients, a fibrous capsule forms around the implant (ie, encapsulation). The capsule may be soft and nonpalpable or hard and resistant. Two types of silicone gel-filled breast implant ruptures can occur: intracapsular rupture occurs when silicone escapes the elastic membrane shell but is contained in the fibrous capsule. This form of silicone gel-filled breast implant rupture is most common. Extracapsular rupture involves the escape of free silicone gel through the fibrous capsule, with extravasation into the breast tissue. Migration of silicone gel to the axillary lymph nodes also may be present. Furthermore, silicone gel can migrate to the brachial plexus, chest wall, axilla and the wrist.

To reduce implant rupture, improvements have been made on the structure of the implant envelope or shell. U.S. Pat. Nos. 4,455,691 and 4,472,226 disclose a three layer implant wall comprising a middle layer made of a heteropolymer of dimethylpolysiloxane and siloxane elastomer, which substantially impedes the migration of silicone gel. Commercially, breast implants constructed with low diffusion silicone elastomer shells are available from the INAMED Corporation, Santa Barbara, Calif. The low diffusion shell has a barrier coat between two layers of silicone elastomer to minimize silicone diffusion. U.S. Pat. No. 5,630,844 discloses another three layer implant shell which comprises a hydrophobic thermoplastic elastomer middle layer as a water vapor barrier, which can be used with a broader scope of filling materials and can reduce ruptures due to fold flaw fracture caused by loss of water vapor from the shell.

Furthermore, a new cohesive silicone gel has been developed and is already in use for breast implants in Canada, Europe and other countries. The cohesive silicone gel is expected to be approved for breast implants in the United States in the near future. Different from the silicone gel traditionally used for breast implants, cohesive silicone gel does not leak out from the shell of the implant. However, when the implant shell ruptures, the patient's tissue will be in contact with the cohesive silicone gel, which can potentially cause inflammation and other effects of silicone to the human body.

The diagnosis of silicone gel-filled breast implant rupture is useful to both clinicians and patients; it aids in surgical decision-making and helps the patient gain peace of mind. Furthermore, the systemic effects of leaked silicone gel-filled breast implants, if any, remain unclear. Currently, magnetic resonance imaging (MRI) is used to evaluate silicone gel-filled breast implants, because the findings at clinical examination often are nonspecific. However, MRI is an expensive examination involving complex instrumentation and data processing.

The above-described problems also present with other implants used in cosmetic and reconstructive surgery using silicone gel as the implant filling material, such as brow, nose, cheek, chin, lips, pectoral, breast, triceps and biceps, genitals, buttocks and calf. Among these, some require a small amount of implant filling material, some require a large amount of filling material. For example, the calf implant is inserted to rebalance legs affected by such diseases as polio, which requires a relatively large amount of filling material. In general, the larger the amount of implant filling material, the worse the potential impact of filling material to a patient can be. Therefore, it is apparent that there exists a need for cost effective and more convenient test methods for detection of the rupture of the silicone gel-filled breast implants and other silicone cosmetic and reconstructive implants.

On the other hand, various biocompatible dyes have been used in pharmaceuticals or food industries for human use. For example, U.S. Pat. No. 6,020,374 teaches various synthetic dye compounds for pharmaceutical uses, such as Aurintricarboxylic acid (ATA), Halogenated ATA, Sulfonated ATA, Sulfonated-Halogenated ATA, Phosphorylated ATA, Anazolene Sodium, Eosine I Bluish, Eosine Yellowish, Erythrosine, Evan's Blue (EB), Fast Green FCF, Fuchin(e) Acid, Iodophthalein Sodium, Rose Bengal, Sulfobromophthalein Sodium, Suramin Sodium, Trypan Blue, Trypan Red, Rosaniline Chloride, Crystal Violet, Methyl Blue, Methyl Green, Coomassie Blue, Basic Fuchsin, Malachite Green, Brilliant Green, Aniline blue, Brilliant Cresyl Blue, Safranin O, Ethyl Violet, Pararosaniline Acetate, Methyl Violet, Direct Yellow, Direct Red, Ponceau S, Ponceau SS, Nitrosulfonazo III, Chicago Sky Blue 6B, and Calcion or RG-13577.

Phenazopyridine hydrochloride, formally 2,6-Pyridinediamine, 3-(phenylazo)-, monohydrochloride, is an oral medication that has been clinically used for treating urinary tract discomfort for many years. Phenazopyridine hydrochloride relieves urinary tract pain, burning, irritation, and discomfort, as well as urgent and frequent urination caused by urinary tract infections, surgery, injury, or examination procedures. It comes as a tablet, and the usual dosage is 200 mg three times a day (about 4 mg/kg). The medicine is metabolized in the liver and other tissues and excreted in the urine. Nearly 90% of an oral dose is excreted renally through the kidney in 24 hours. It turns the urine to a reddish-orange color.

The above-described biocompatible dyes, methylene blue and phenazopyridine hydrochloride have not been utilized for indicating or detecting a rupture of breast or other implants.

### SUMMARY OF THE INVENTION

In one aspect, the present invention is directed to a prosthesis containing a rupture indicator, which comprises an indicator lumen enclosed by an indicator lumen envelope made of at least one layer of a first elastomer containing therein a biologically compatible chemical indicator for indicating rupture of the prosthesis and a carrier medium; and at least one implant lumen enclosed by an implant lumen envelope made of at least one layer of a second elastomer, disposed within the indicator lumen. The implant lumen contains therein an implant filling material.

In one embodiment, the present invention provides a double lumen prosthesis which comprises an external envelope made of at least one layer of a first elastomer containing therein a fluid material and a biologically compatible chemical indicator for indicating rupture of the prosthesis, and an internal envelope made of at least one layer of a second elastomer, disposed within the external envelope. The internal envelope contains therein an implant filling material. In one embodiment, the internal or external envelope, or both, can be multi-layered to enhance the strength of the envelope and reduce diffusion of silicone filling material.

The biologically compatible chemical indicator can be phenazopyridine hydrochloride, or a dye, such as methylene blue and various other dyes described in detail in the specification; an odour generating agent which generates a non-human body smell or taste when leaking out from the prosthesis.

In a further embodiment, the prosthesis comprises an internal implant lumen disposed within an external implant lumen, each implant lumen filled with an implant filling material, and an indicator lumen disposed at the most exterior of the prosthesis. In an alternative embodiment, the indicator lumen is disposed outside the internal implant lumen and within the external implant lumen.

The prosthesis containing a rupture indicator can be breast, brow, nose, cheek, chin, lips, pectoral, triceps and biceps, genitals, buttocks and calf prosthesis, used for cosmetic and reconstructive surgeries.

In a further aspect, the present invention is directed to a method of detecting rupture of a prosthesis used in cosmetic and reconstructive procedures. The method comprises surgically implanting a prosthesis containing a biologically compatible chemical indicator for indicating rupture of the prosthesis in a location of a patient's body in need of the prosthesis; and detecting a change of a body excretion, secretion, or peripheral blood for indication of leakage of the indicator from the prosthesis. The body excretion or secretion that can be used for the detection includes materials such as urine, saliva, perspiration and feces. The changes include a presence of the chemical indicator or metabolized product thereof in the body excretion, secretion, or peripheral blood, an odour from the indicator in the body excretion or secretion, a color change of at least one of the body's excretion or secretion, and a change in sensation or taste caused by the presence of the indicator in the body secretion.

Furthermore, the method of detecting rupture of a prosthesis includes detecting a change locally around the prosthesis for indication of leakage of the indicator from the prosthesis. The change includes a local skin color change, and a local x-ray opacity change from that after the surgically implanting the prosthesis.

In yet a further embodiment, the present invention is directed to a single lumen prosthesis which comprises an envelope made of at least one layer of an elastomer containing therein an implant filling material and a biocompatible chemical indicator in a carrier medium for indicating rupture of the prosthesis.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a side view of a double lumen breast prosthesis in one embodiment of the present invention, which has an external envelope containing therein a biologically compatible chemical indicator in a carrier medium and an internal envelope filled with an implant filling material.

FIG. 2 is a side view of a triple lumen breast prosthesis in a further embodiment of the present invention, which has an internal implant lumen disposed within an external implant lumen and an indicator lumen disposed at the most exterior of the prosthesis.

FIG. 3 is a side view of a triple lumen breast prosthesis in another embodiment of the present invention, which has the indicator lumen disposed outside the internal implant lumen and within the external implant lumen.

FIG. 4 is a side view of the double lumen breast prosthesis shown in FIG. 1, which further includes a filling tube.

FIG. 5 is a side view of a single lumen breast prosthesis in a further embodiment of the present invention, which is enclosed by an envelope containing therein an implant filling material and a chemical indicator in a carrier solution.

### DETAILED DESCRIPTION OF THE INVENTION

In one embodiment, the present invention provides a prosthesis containing a rupture indicator. The prosthesis comprises an external lumen enclosed by an external envelope made of at least one layer of an elastomer containing therein a biologically compatible chemical indicator for indicating rupture of the prosthesis and a carrier material, and an internal lumen enclosed by an internal envelope made of at least one layer of an elastomer containing therein an implant filling material. The internal lumen is disposed within the external lumen. It is noted that the term of envelope used herein is also commonly referred to as shell.

The rupture of a prosthesis is defined herein as the development of a tear or a hole in the envelope or shell of the prosthesis. A range of rupture characteristics that have been reported in the literature are included, from foci involving very small holes with a very small amount of silicone gel present outside of the envelope or shell, to larger visible physical tears and complete destruction with the prosthesis envelope or shell surrounded by silicone gel.

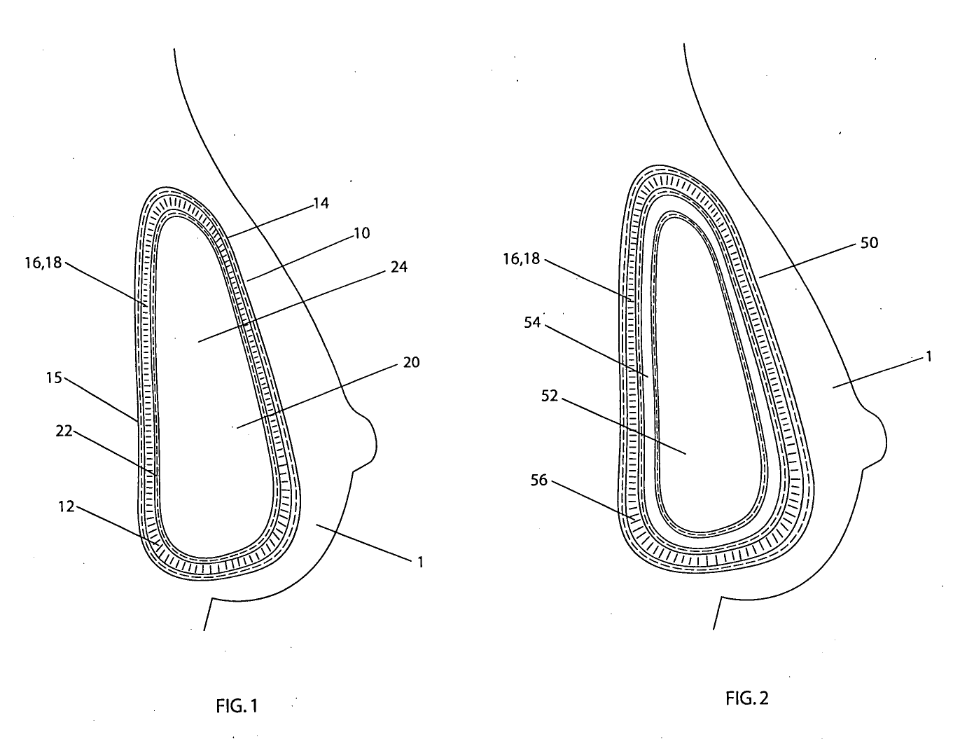

As shown in FIG. 1, a breast prosthesis 10 implanted in a human breast 1 includes an external lumen 12 enclosed by an external envelope 14. The external lumen 12 is filled with a biologically compatible chemical indicator 18 in a carrier medium 16 (shown by cross hatching). Preferably the carrier medium is a fluid material which has a low viscosity such as an aqueous solution. The breast prosthesis 10 also includes an internal lumen 20 enclosed by an internal envelope 22. The internal lumen 20 is filled with an implant filling material 24, preferably a material having a much higher viscosity such as a silicone gel.

Suitable examples of implant filling materials include, but are not limited to, glycosaminoglycan including hyaluronic acid, chondroitin 4-sulfate, chondroitin 6-sulfate, dermatan sulfate, heparin sulfate, and keratan sulphate; mucopolysaccharide, polyvinylpyrollidone, polyvinyl pyrralidone, polyvinyl alcohol, polyacrlimides, polysaccharides, hydroxypropylmethyl cellulose, polyethylene oxide, hyaluronic acid, sodium or calcium alginate, hydrogel polyurethane, hydroxyethyl starch, polyglycolic acid, polyacrylamide, hydroxyethylmethacrylate (HEMA), and naturally derived biopolymers including sodium kinate, seaweed, and agar; aqueous solution of polyethylene glycol; linear or branched, or cross-linked polyacrylamide, sodium hyaluronate, phosphatidylcholine (PC), hydroxypropylmethyl cellulose (HPMC) and its derivatives including hydroxyalkyl cellulose, sodium carboxymethyl cellulose, hydroxyethyl cellulose, hydroxypropyl cellulose, methylhydroxypropyl cellulose, methyl cellulose and ethylhydroxyethyl cellulose; and polyoxyethylene/polyoxypropylene block copolymers which have gelling properties at body temperature. Furthermore, the implant filling material can also be a saline solution.

Suitable examples of the carrier medium include, but are not limited to, aqueous solution, physiological saline solution, oil, water soluble gel, and other biocompatible fluid materials. Preferably, the carrier medium is isotonic.

The external envelope 14 and internal envelope 22 are made of at least one layer of a soft flexible biocompatible material such as a silicone elastomer. Suitable materials include, but are not limited to, silicone elastomers such as, polydimethylsiloxane, polymethylvinylsiloxane, copolymers thereof, or heterpolymers of diphenylpolysiloxane and dimethylpolysiloxane having diphenyl polysiloxane substituents. Other polymers may be substituted as will be apparent to those skilled in the art. The external envelope 14 and internal envelope 22 can be made of the same material or different materials.

In one embodiment, the internal envelope 22, or external envelope 14, or both, can be constructed of two or three layers of silicone elastomer to reduce silicone diffusion and enhance the strength of the envelope. One suitable example has been described in U.S. Pat. No. 4,455,691, which is herein incorporated by reference in its entirety. More specifically, the envelope can be made of three layers, with inner and outer layers made of the silicone elastomer described above, and a middle layer in-between functioning as a barrier to silicone diffusion. The middle layer can be made of a reaction product of dimethylpolysiloxane and siloxane elastomer such as 3,3,3-trifluoropropylpolysiloxane, diphenylpolysiloxane, or methylphenylpolysiloxane. It is noted that the elastomer used for the inner/outer layers of the internal envelope can be the same as the material used for the external envelope, and can also be different.

Furthermore, the commercially available material, known as low diffusion silicone elastomer shell produced by the INAMED Corporation (Santa Barbara, Calif.), can be used for construction of the internal and external envelopes for the purpose of the present invention. The low diffusion silicone elastomer shell is made of two layers of silicone elastomer with a barrier coat between the two layers. The barrier coat can be the reaction product of dimethylpolysiloxane and siloxane elastomer as described above.

In another embodiment, the external envelope 14 can have a multi-layer structure comprising a hydrophobic thermoplastic elastomer layer which functions as a water vapour barrier for maintaining the aqueous filling material at the desired osmotic balance in the envelope. This effect can reduce implant rupture through fold flaw fracture caused by loss of water vapour. The materials and the process of making the multi-layer envelope has been described in detail in U.S. Pat. No. 5,630,844, which is herein incorporated by reference in its entirety. More specifically, the inner and outer layers can be made of silicone elastomer, and the middle layer is made of a thermoplastic elastomer such as styrene block copolymers, or a mixture of styrene block copolymers and ethylene-propylene based copolymers which are thermoplastic elastomers. Preferably, the thermoplastic elastomers have triblock copolymers with styrene end-blocks and a mid-block of an elastomer polymer selected from olefin, vinyl, and dienyl based polymers.

As illustrated, the external envelope 14 has a generally tear-drop shape with a relatively flat rear portion 15 and rounded dome or a forward surface 17. The external envelope 14 defines an external lumen which may be of a generally tear-drop shape or other non-symmetrical shape in order to conform to the contours of a human breast. It should be recognized that in certain cases a round shape may be desirable.

The biologically compatible chemical indicator can be several types. One type of chemical indicators causes a color change of the body excretion or secretion. Suitable examples include, but are not limited to, phenazopyridine hydrochloride, or biocompatible dyes such as aurintricarboxylic acid (ATA), halogenated ATA, sulfonated ATA, sulfonated-halogenated ATA, phosphorylated ATA, anazolene sodium, eosine I bluish, eosine yellowish, erythrosine, Evan's blue (EB), fast green FCF, fuchin(e) acid, iodophthalein sodium, rose bengal, sulfobromophthalein sodium, suramin sodium, trypan blue, trypan red, rosaniline chloride, crystal violet, methyl blue, methyl green, methylene blue, coomassie blue, basic fuchsin, malachite green, brilliant green, aniline blue, brilliant cresyl blue, safranin O, ethyl violet, pararosaniline acetate, methyl violet, direct yellow, direct red, ponceau S, ponceau SS, nitrosulfonazo III, chicago sky blue 6B, calcion or RG-13577, and commonly used food dyes such as FD&C red No. 3, FD&C red No. 40, FD&C blue No. 1 and FD&C yellow No. 5.

In a preferred embodiment, phenazopyridine hydrochloride is used. As described previously, the majority of phenazopyridine hydrochloride administered systemically excretes in 24 hours, and the excreted phenazopyridine hydrochloride changes urine color to reddish-orange. This property can be used to indicate timely a rupture of the prosthesis to the user.

Furthermore, the dye used for the purpose of the present invention is preferably water soluble so that it can release out through body excretion or secretion, such as urine, saliva, perspiration, and feces, or in peripheral blood when the prosthesis ruptures. When the prosthesis ruptures, even a minor rupture, chemical indicator 18 leaks out from external lumen 12 into the tissues where it is absorbed into the vascular system of the body. Optionally, chemical indicator 18 can also be contained in the internal lumen 20, which will leak out when both envelopes rupture. In one embodiment, the leaked chemical indicator 18 can be visually detected in urine, or saliva. It can also be detected in a body excretion or secretion sample or a peripheral blood sample using a colorimetric method. Such detection can be performed in a clinical laboratory, or can be performed using a specifically designed kit for home use, similar to the glucose, or pregnancy test kits. The Example described hereinafter provides a detailed configuration of the breast implant of the present invention and the method of detection. In an exemplary embodiment of a breast implant, the filling material in the internal lumen is 85% or more of the total volume of the prosthesis for maintaining the overall prosthesis properties, and the fluid material in the external lumen is 15% or less. The ratio between the filling material and the fluid material in the external lumen can be different for different types of prostheses.

With water soluble dyes, the rupture can also be detected by staining of skin locally by the leaked dye. Furthermore, in addition to dyes, other non-coloring biocompatible chemical indicators, detectable at a trace amount, can also be used, which can be detected in body excretion or secretion, such as urine, saliva, perspiration and feces, or in peripheral blood, using a chemical reaction which is sensitive and specific to the indicator.

Another type of the biologically compatible chemical indicators is an odour generating material which causes a smell change of body excretion or secretion, such as saliva, urine, perspiration and feces, or a taste change. One example is a sterilized garlic solution. When the breast prosthesis ruptures, the odour generating solution leaks into the tissues where it is absorbed into the vascular system, and subsequently causes an unusual body odour, hence, alert the user.

A further type of chemical indicator is a material which causes a temporary local tissue x-ray opacity. Using this type indicator, a simple mammogram at annual routine examination of a user can detect the leak from the rupture.

Although the prosthesis and the method of use of the present invention have been described above with a double lumen breast implant structure, it should be understood that other implant structures can also be used with the biologically compatible chemical indicators described above. In general, the prosthesis comprises an indicator lumen enclosed by an indicator lumen envelope made of at least one layer of an elastomer containing therein a biologically compatible chemical indicator for indicating rupture of the prosthesis and a carrier medium; and at least one implant lumen enclosed by an implant lumen envelope made of at least one layer of an elastomer, disposed within the indicator lumen, wherein the implant lumen containing therein an implant filling material. Herein, for the purpose of description of the instant invention, the term “implant lumen” denotes a lumen being filled with an implant filling material, such as a silicone gel, or a saline solution, without a rupture indicator. Preferably, the indicator lumen is disposed at the most exterior of the prosthesis.

It is apparent that the prosthesis structure illustrated in FIG. 1 is one specific example, with one implant lumen disposed with the indicator lumen. As another example shown in FIG. 2, the prosthesis 50 comprises an internal implant lumen 52 disposed within an external implant lumen 54, and an indicator lumen 56 disposed outside the external implant lumen, with each lumen enclosed by its envelope. In this case, the prosthesis has a triple lumen structure, with the most exterior lumen as the indicator lumen. The internal and external implant lumens can be filled with a same or different implant filling materials, wherein these two lumens can be structured either in the form of the traditional double lumen or in the form of the reversed double lumen breast prosthesis known in the breast implant industry. Since the indicator lumen is located at the most exterior of the prosthesis, when the prosthesis ruptures, the chemical indicator releases into the tissues where it is absorbed and subsequently indicates the rupture in one of the mechanisms described above.

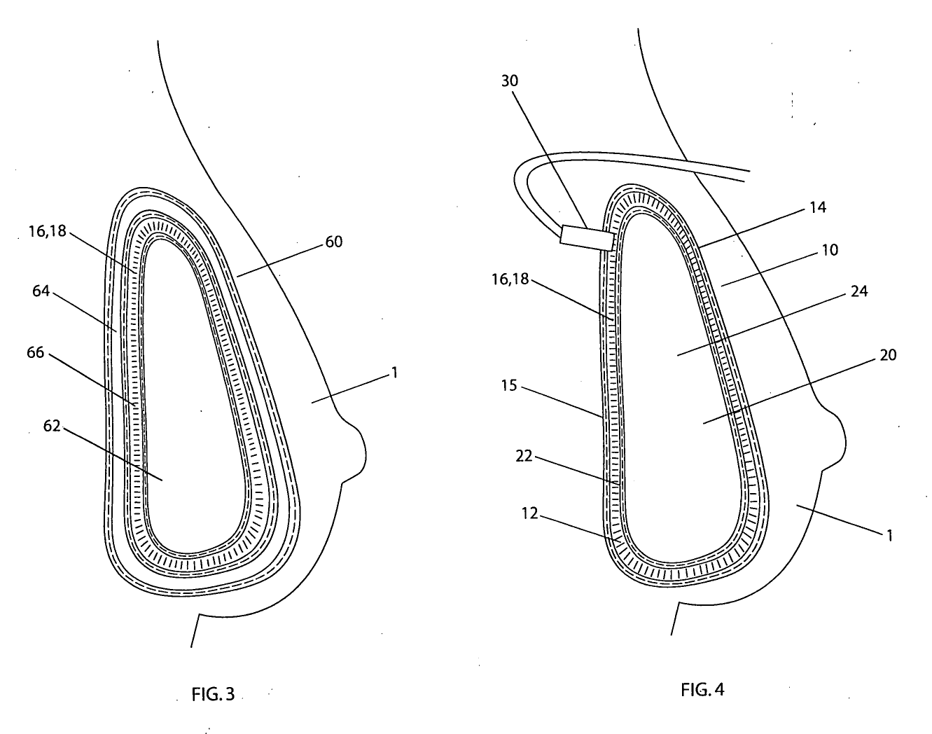

In an alternative embodiment as shown in FIG. 3, the breast prosthesis 60 has the indicator lumen 66 disposed outside the internal implant lumen 62 and within the external implant lumen 64. This structural arrangement is suitable for the traditional double lumen breast implant, wherein the internal implant lumen 62 is filled with a silicone gel, and the external implant lumen 64 is filled with a saline solution. In this situation, when the chemical indicator releases into the body, it indicates the rupture or damage of the external implant lumen, and a potential damage of the internal implant lumen envelope.

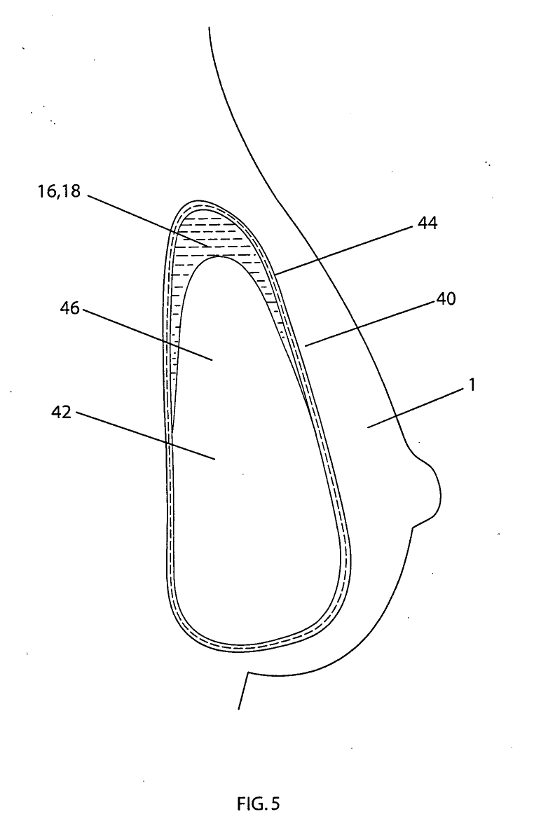

A further embodiment of the present invention includes means for adding or removing the chemical indicator 18 in the carrier medium 16 to or from the external lumen 12 and/or the implant filling material 24 to or from internal lumen 20. One such means is illustrated in FIG. 4. As shown, a filling tube 30 is in an inserted position within the external lumen 12 and can be inserted at the time of manufacture. Alternatively, a filling tube can be inserted later. The filling tube 30 is typically inserted through a self-sealing valve (not shown) commonly used in breast implant surgery. The distal end of filling tube 30 is connected with a source of the chemical indicator or implant filling material. Upon completion of the filling process, the filling tube 30 is removed and the self-sealing valve closes. Furthermore, other filling valves currently used in the breast implant industry, such as the filler valve on the Becker Expandable breast prosthesis by Mentor Corporation, Santa Clara, Calif., can also be incorporated into the prosthesis structure of the present invention.

Using the breast prosthesis containing a rupture indicator and the method of detection, the potential rupture of the breast prosthesis can be conveniently detected. With the present invention, an early detection of the rupture is possible. Since when chemical indicator contained in the external lumen 12 leaks out, it indicates a potential problem of the breast prosthesis, even if the internal envelope has not ruptured. A further confirmation examination can be performed using MRI.

In another embodiment, the present invention provides a single lumen prosthesis containing a rupture indicator. As shown in FIG. 5, a breast prosthesis 40 implanted in a human breast 1 includes single lumen 42 enclosed by an envelope 44. The single lumen 42 is filled with an implant filling material 46, such as silicone gel or other suitable filling materials as described above, and a biologically compatible chemical indicator 18 in a carrier medium 48. Suitable chemical indicators have been described above. Preferably, a water soluble chemical indicator is used with an aqueous solution as the carrier medium so that when the breast prosthesis ruptures the chemical indicator releases out into the tissues and then is absorbed into the vascular system. It is noted that the relative position of the implant filling material 46 versus the position of the carrier medium 48 can vary depending on the densities of the filing material and the carrier medium, as well as the position of the body. In other words, the implant filling material can be either above or below the carrier medium containing the chemical indicator.

The carrier medium 48 can be an aqueous solution such as a saline solution, and can further contain an antimicrobial as preservative. Moreover, the carrier medium can further contain a surfactant. The surfactant in the carrier medium forms micelles which attract and maintain the organic indicator molecules in the carrier medium.

In an exemplary embodiment, single lumen 42 contains 85% or more in volume of the filling material 46 and 15% or less in volume of the chemical indicator in the carrier medium.

### EXAMPLE 1

A double lumen breast implant having a structure shown in FIG. 1 has a silicone gel commonly used in the breast implant as the filling material inside the internal lumen 20. The external lumen contains from about 35 to about 45 ml of sterilized aqueous solution of methylene blue. The methylene blue is in a concentration range from about 1 mg/ml to about 4 mg/ml. With the concentration and volume of the methylene blue described, it is in a range from about 1 to about 2 mg per kilogram of body weight for an average female (from about 50 to about 70 kg). In the event of rupture, the methylene blue solution leaks out from the external lumen into the tissues where it is absorbed into the vascular system, metabolizes in kidney, and releases to urine, which causes a color change of the urine.

### EXAMPLE 2

A double lumen breast implant is constructed having a general structure shown in FIG. 1. Both internal and external envelopes are made of silicone elastomer currently used for breast implant. More specifically, the internal envelope 22 can be constructed of the low diffusion shell produced by INAMED Corporation (Santa Barbara, Calif.). The internal lumen 20 is filled with a cohesive silicone gel currently used in breast implants in some countries. The external lumen 12 contains from about 35 to about 45 ml of sterilized aqueous solution of phenazopyridine hydrochloride. The phenazopyridine hydrochloride is in a concentration range from about 2 mg/ml to about 17 mg/ml. With the concentration and volume of the phenazopyridine hydrochloride described, it is in a range from about 1.4 to about 12 mg per kilogram of body weight for an average female (from about 50 to about 70 kg). In the event of rupture, the phenazopyridine hydrochloride solution leaks out from the external lumen into the tissues where it is absorbed into the vascular system, and releases to urine, which causes a reddish-orange color of the urine.

The biocompatible chemical indicators and the method of detection of implant rupture are specifically described using breast prosthesis. It should be understood, however, the materials and the methods can also be used for other cosmetic and reconstructive prostheses, such as brow, nose, cheek, chin, lips, pectoral, triceps and biceps, genitals, buttocks and calf.

While the present invention has been described in detail and pictorially shown in the accompanying drawings, these should not be construed as limitations on the scope of the present invention, but rather as an exemplification of preferred embodiments thereof. It will be apparent, however, that various modifications and changes can be made within the spirit and the scope of this invention as described in the above specification and defined in the appended claims and their legal equivalents.

1. A prosthesis containing a rupture indicator comprising:
(a) an indicator lumen enclosed by an indicator lumen envelope made of at least one layer of a first elastomer, containing therein a biologically compatible chemical indicator for indicating rupture of said prosthesis and a carrier medium; and
(b) at least one implant lumen enclosed by an implant lumen envelope made of at least one layer of a second elastomer, disposed within said indicator lumen; said implant lumen containing therein an implant filling material.

2. The prosthesis containing a rupture indicator of claim 1, wherein said biologically compatible chemical indicator is phenazopyridine hydrochloride.

3. The prosthesis containing a rupture indicator of claim 1, wherein said biologically compatible chemical indicator is methylene blue.

4. The prosthesis containing a rupture indicator of claim 1, wherein said biologically compatible chemical indicator is at least one selected from the group consisting of aurintricarboxylic acid (ATA), halogenated ATA, sulfonated ATA, sulfonated-halogenated ATA, phosphorylated ATA, anazolene sodium, eosine I bluish, eosine yellowish, erythrosine, Evan's blue (EB), fast green FCF, fuchin(e) acid, iodophthalein sodium, rose bengal, sulfobromophthalein sodium, suramin sodium, trypan blue, trypan red, rosaniline chloride, crystal violet, methyl blue, methyl green, coomassie blue, basic fuchsin, malachite green, brilliant green, aniline blue, brilliant cresyl blue, safranin O, ethyl violet, pararosaniline acetate, methyl violet, direct yellow, direct red, ponceau S, ponceau SS, nitrosulfonazo III, chicago sky blue 6B, calcion or RG-13577, FD&C red No. 3, FD&C red No. 40, FD&C blue No. 1 and FD&C yellow No. 5.

5. The prosthesis containing a rupture indicator of claim 1, wherein said biological compatible chemical indicator is an odour generating agent which generates a smell or a taste when leaking out from said prosthesis.

6. The prosthesis containing a rupture indicator of claim 1, wherein said carrier medium is a fluid material.

7. The prosthesis containing a rupture indicator of claim 6, wherein said carrier medium is an aqueous solution.

8. The prosthesis containing a rupture indicator of claim 1, wherein said implant filling material is a silicone gel.

9. The prosthesis containing a rupture indicator of claim 1 wherein said implant filling material is a saline solution.

10. The prosthesis containing a rupture indicator of claim 1, wherein said indicator lumen is disposed at the most exterior of said prosthesis.

11. The prosthesis containing a rupture indicator of claim 1, wherein said at least one implant lumen includes an internal implant lumen enclosed by an internal implant lumen envelope, disposed within an external implant lumen which is enclosed by an external implant lumen envelope.

12. The prosthesis containing a rupture indicator of claim 11, wherein said indicator lumen is disposed outside said external implant lumen.

13. The prosthesis containing a rupture indicator of claim 11, wherein said indicator lumen is disposed outside said internal implant lumen and within said external implant lumen.

14. The prosthesis containing a rupture indicator of claim 13, wherein said implant filling material contained in said internal implant lumen is a silicone gel.

15. The prosthesis containing a rupture indicator of claim 16, wherein said implant filling material contained in said external implant lumen is a saline solution.

16. The prosthesis containing a rupture indicator of claim 1, wherein said indicator lumen further comprises a filling means for filling said biologically compatible chemical indicator and said carrier medium.

17. The prosthesis containing a rupture indicator of claim 1, wherein said envelopes comprise a first inner layer and a first outer layer, both made of said first elastomer, and a first barrier layer between said first inner and first outer layers.

18. The prosthesis containing a rupture indicator of claim 1, wherein said indicator lumen envelope comprises a first inner layer and a first outer layer, both made of said first elastomer, and a first barrier layer between said first inner and first outer layers.

19. The prosthesis containing a rupture indicator of claim 1, wherein said implant lumen envelope comprises a second inner layer and a second outer layer, both made of said second elastomer, and a second barrier layer between said 25 second inner and second outer layers.

20. The prosthesis containing a rupture indicator of claim 1, wherein said first and second barrier layers are made of a same material.

21. The prosthesis containing a rupture indicator of claim 1, wherein said first and second barrier layers are made of different materials.

22. The prosthesis containing a rupture indicator of claim 1, wherein said prosthesis is a breast prosthesis.

23. The prosthesis containing a rupture indicator of claim 1, wherein said prosthesis is at least one selected from the group consisting of brow, nose, cheek, chin, lips, pectoral, triceps and biceps, genitals, buttocks and calf prostheses.

24. A method of detecting rupture of a prosthesis comprising:
(a) surgically implanting the prosthesis of claim 1 in a location of a patient's body in need of said prosthesis; and
(b) detecting a change of a body excretion or secretion or peripheral blood for indication of leaking out of said chemical indicator from said prosthesis.

25. The method of claim 24, wherein said body excretion or secretion is at least one selected from the group consisting of urine, saliva, perspiration and feces.

26. The method of claim 24, wherein said change is a presence of said chemical indicator or metabolized product thereof in said body excretion or secretion, or peripheral blood.

27. The method of claim 24, wherein said change is an odour from said indicator in said body excretion or secretion.

28. The method of claim 24, wherein said change is a color change of at least one of said body excretion or secretion.

29. A method of detecting rupture of a prosthesis comprising:
(a) surgically implanting a prosthesis of claim 1 in a location of a patient's body in need of said prosthesis; and
(b) detecting a change locally around said prosthesis for indication of leaking out of said chemical indicator from said prosthesis.

30. The method of claim 29, wherein said change is a local skin color change.

31. The method of claim 29, wherein said change is a local x-ray opacity change from that after said surgically implanting said prosthesis.

32. A prosthesis containing a rupture indicator comprising a lumen enclosed by an envelope made of at least one layer of an elastomer, said lumen containing therein an implant filling material and a biocompatible chemical indicator in a carrier medium for indicating rupture of said prosthesis.

33. The prosthesis of claim 32, wherein said carrier medium is an aqueous solution.

34. The prosthesis of claim 32, wherein said implant filling material is a silicone gel.

35. The prosthesis of claim 32, wherein said implant filling material is a saline solution.

36. The prosthesis containing a rupture indicator of claim 32, wherein said biologically compatible chemical indicator is phenazopyridine hydrochloride.

37. The prosthesis containing a rupture indicator of claim 32, wherein said biologically compatible chemical indicator is methylene blue.

38. The prosthesis containing a rupture indicator of claim 32, wherein said biologically compatible chemical indicator is at least one selected from the group consisting of aurintricarboxylic acid (ATA), halogenated ATA, sulfonated ATA, sulfonated-halogenated ATA, phosphorylated ATA, anazolene sodium, eosine I bluish, eosine yellowish, erythrosine, Evan's blue (EB), fast green FCF, fuchin(e) acid, iodophthalein sodium, rose bengal, sulfobromophthalein sodium, suramin sodium, trypan blue, trypan red, rosaniline chloride, crystal violet, methyl blue, methyl green, coomassie blue, basic fuchsin, malachite green, brilliant green, aniline blue, brilliant cresyl blue, safranin O, ethyl violet, pararosaniline acetate, methyl violet, direct yellow, direct red, ponceau S, ponceau SS, nitrosulfonazo III, chicago sky blue 6B, calcion or RG-13577, FD&C red No. 3, FD&C red No. 40, FD&C blue No. 1 and FD&C yellow No. 5.

39. The prosthesis containing a rupture indicator of claim 32, wherein said envelope comprises an inner layer and an outer layer made of said elastomer and a barrier layer between said inner and outer layers.

40. A method of detecting rupture of a prosthesis comprising:
(a) surgically implanting the prosthesis of claim 32 in a location of a patient's body in need of said prosthesis; and
(b) detecting a change of a body excretion or secretion or peripheral blood for indication of leaking out of said chemical indicator from said prosthesis.

41. The method of claim 40, wherein said body excretion or secretion is at least one selected from the group consisting of urine, saliva, perspiration and feces.

42. The method of claim 40, wherein said change is a presence of said chemical indicator or metabolized product thereof in said body excretion or secretion, or peripheral blood.

43. The method of claim 40, wherein said change is an odour from said indicator in said body excretion or secretion.

44. The method of claim 40, wherein said change is a color change of at least one of said body excretion or secretion.

45. A method of detecting rupture of a prosthesis comprising:
(a) surgically implanting a prosthesis of claim 32 in a location of a patient's body in need of said prosthesis; and
(b) detecting a change locally around said prosthesis for indication of leaking out of said chemical indicator from said prosthesis.

46. The method of claim 45, wherein said change is a local skin color change.

47. The method of claim 45, wherein said change is a local x-ray opacity change from that after said surgically implanting said prosthesis.
